(19) United States
(12) Patent Application Publication
(10) Pub. No.: US 2004/0243062 A1
(43) Pub. Date: Dec. 2, 2004

(54) Trocar assembly and method

(76) Inventor: Robert E. Henry, Nashville, TN (US)

Correspondence Address: GEORGE A. COURY, BACHMAN & LaPOINTE, P.C., Suite 1201, 900 Chapel Street, New Haven, CT, 06510-2802, US

(21) Appl. No.: 10/178,661

(22) Filed: Jun. 21, 2002

(51) Int. Cl. A61M 5/178

(52) U.S. Cl. 604/164.13

(57) ABSTRACT

A method for positioning a trocar in a body cavity, includes the steps of positioning a guide wire through a body cavity wall into a body cavity; positioning a trocar over the guide wire; and sliding the trocar over the guide wire and through the body cavity wall, whereby the trocar is guided by the guide wire.

### BACKGROUND OF THE INVENTION

The invention relates to a trocar assembly and method for safely positioning same through a body cavity wall into a body cavity.

In laparascopic or endoscopic surgery, it is frequently necessary to pierce the body wall with a trocar and position a cannula or port through the incision in the body wall. This leads to risk of injury, for example to underlying organs, tissues and the like, as the trocar or piercing member is forced through the body wall.

Efforts to avoid injury to underlying organs and the like include shields disposed over the trocar tip and/or insufflating the body cavity so as to lift the abdomen wall away from underlying organs. Nevertheless, the risk remains of injury to the underlying organs.

It is therefore the primary object of the present invention to provide a trocar assembly and method whereby the trocar is safely positioned through the abdomen wall.

Other objects and advantages will appear hereinbelow.

### SUMMARY OF THE INVENTION

In accordance with the present invention, the foregoing object has been readily attained.

In accordance with the invention, a method is provided for positioning a trocar through an abdomen wall, which method comprises the steps of positioning a guide wire through a body cavity wall into a body cavity; positioning a trocar over said guide wire; and sliding said trocar over said guide wire and through said body cavity wall, whereby said trocar is guided by said guide wire.

### BRIEF DESCRIPTION OF THE DRAWINGS

A detailed description of preferred embodiments of the present invention follows, with reference to the attached drawings, wherein:

FIGS. 1-4 schematically illustrate the trocar and method in accordance with the present invention; and

FIG. 5 illustrates a trocar and cannula in accordance with the present invention.

### DETAILED DESCRIPTION

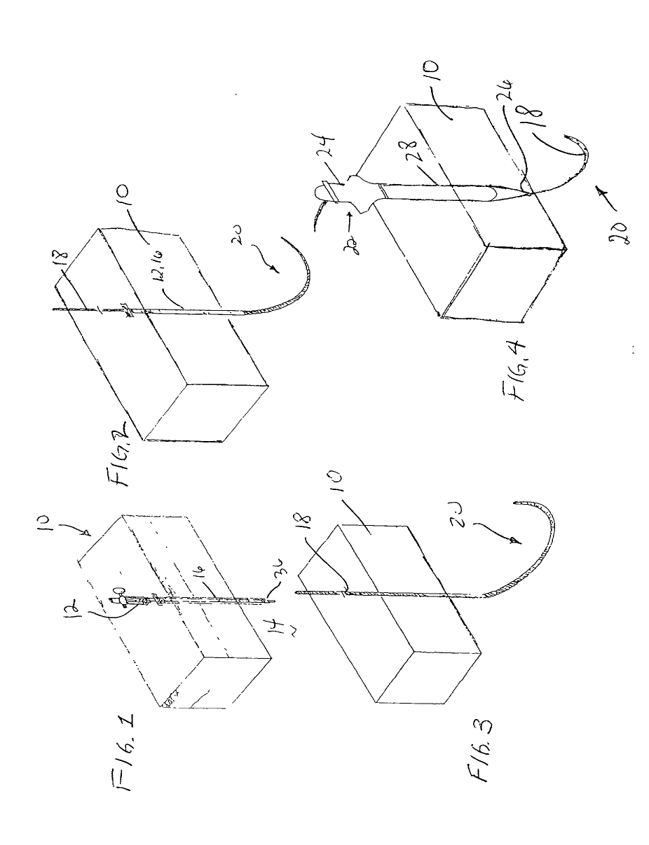

Turning to FIGS. 1-4, the trocar assembly and method of the present invention are further illustrated. FIG. 1 shows an abdomen wall 10 through which it is desired to place a cannula or sheath in order to conduct a laparascopic or endoscopic procedure. An insufflation/access needle 12 is positioned through abdominal wall 10 and into the abdominal cavity 14 to be accessed. Following insertion, the abdomen or body cavity is insufflated, typically with CO₂ gas, so as to distend the abdomen wall away from underlying organs, tissues and the like.

Insufflation access needle 12 typically may include a sheath 16 positioned thereover, and after insufflation, access needle 12 is removed from access sheath 16 and a guide wire 18 is inserted through access sheath 16. Guide wire 18 is illustrated in FIG. 2 and preferably is provided having a soft or resilient tip or pigtail portion 20 which preferably is provided having resilient shape toward a substantially J-shaped configuration. The soft J-shaped tip is atraumatic, and therefore cannot puncture or otherwise significantly injure internal organs and tissues. As the guide wire is inserted toward the position of FIG. 2, it should enter the distended abdominal cavity with ease. If the patient has adhesions or other obstructions to easy entry, guide wire 18 will not advance and the physician will then know that a different approach is needed. This is a substantial improvement over conventional devices, wherein this type of accurate feedback and information cannot be obtained by the physician, and obtaining this information allows the physician to have a very safe entry into the abdominal cavity.

FIGS. 1 and 2 illustrate the initial steps of the method of the present invention using an access needle 12 having an access sheath positioned thereon. After insufflation, access needle 12 is removed and access sheath 16 is used for positioning guide wire 18 through the body cavity wall as desired. In accordance with an alternative embodiment of the present invention, it may be desirable to provide an access needle 12 through which guide wire 18 can be positioned. This avoids the need for access sheath 16, and therefore simplifies the method and apparatus in accordance with the present invention.

Referring now to FIG. 3, once guide wire 18 is fully inserted, access needle 12 and/or sheath 16 is/are removed leaving only the guide wire extending through abdomen wall 10. Turning to FIG. 4, a trocar assembly 22 is then advantageously positioned through abdomen wall 10 by advancing trocar assembly 22 over guide wire 18.

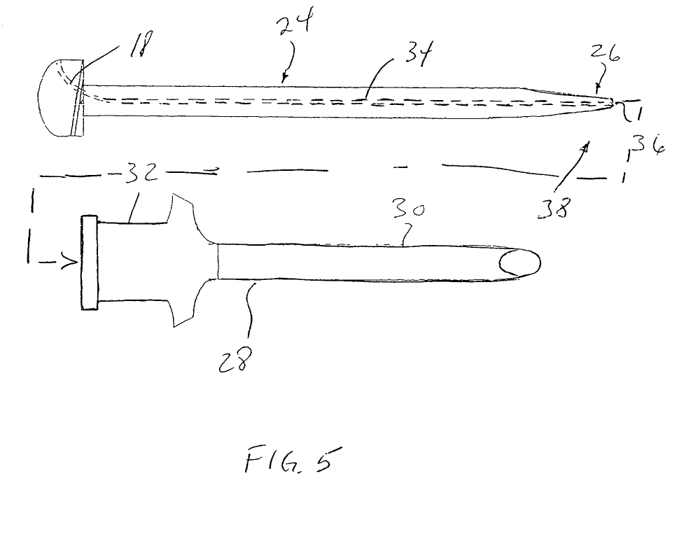

Referring to FIG. 5, a suitable trocar assembly in accordance with the present invention is illustrated, and includes a trocar or trocar obturator portion 24 which is typically an elongate member having a sharp obturator tip 26 which is used for penetrating abdomen walls and the like. Trocar assembly 22 also preferably includes a cannula or sheath member 28 which is also an elongate member having a tube portion 30 through which surgical instruments, materials, fluids and the like can be introduced into and removed from the abdominal cavity, and a valve portion 32 which contains various valves for allowing substantially sealing entry of various instruments and the like, and which further typically has releasable locking structure for releasably locking to trocar obturator 24.

FIG. 5 shows in hidden lines a channel 34 disposed through trocar obturator 24 through which guide wire 18 is advantageously passed and slidably received through trocar obturator 24 such that guide wire 18 can advantageously guide trocar obturator 24, and especially obturator tip 26, in a direction which will not penetrate organs, tissues and the like.

Returning to FIG. 4, trocar assembly 22 is advantageously fed over or along guide wire 18, forced through abdomen wall 10 where obturator tip 26 slightly enlarges the tract through the abdomen wall, and into place through the abdomen wall as desired. Once in place, trocar obturator 24 and guide wire 18 can be removed, leaving trocar cannula or sheath 28 in place through the abdomen wall as desired.

In accordance with the present invention, insufflation/access needle 12 can be any suitable insufflation/access needle as desired in accordance with the present invention. Most advantageously, insufflation/access needle 12 has an insufflation port 36 in a distal end thereof, and insufflation port 36 is preferably tapered so as to allow guide wire 18 to pass easily through and into the abdominal cavity as desired.

Guide wire 18 may be any suitable resilient yet atraumatic material, preferably having shape memory toward the desired J-shape as shown in the Figures. Further, guide wire 18 is preferably sized to readily fit through insufflation/access needle 12, and may for example have a diameter of approximately 0.039 millimeter.

Returning to FIG. 5, trocar assembly 22 contains many conventional elements, for example sheath or cannula 28 and valves contained therein. Trocar obturator 24, however, is advantageously provided having channel 34 which allows for trocar assembly 22 to be positioned over guide wire 18 and used advantageously in accordance with the method of the present invention. In this regard, FIG. 5 shows channel 34 traveling along the elongate member portion of trocar obturator 24. The most important aspect of this configuration is that guide wire 18 extends from obturator tip 26 of trocar obturator 24, and thus that the exit port 36 of channel 34 at a distal end 38 of trocar obturator 24 is advantageously positioned extending from obturator tip 26 as desired.

It should be readily appreciated that the trocar assembly and method in accordance with the present invention advantageously allows for significantly improved safety during placement of a cannula or sheath through an abdomen wall, and will therefore substantially reduce the risk of injury due to underlying adhesions, organs and the like.

It is to be understood that the invention is not limited to the illustrations described and shown herein, which are deemed to be merely illustrative of the best modes of carrying out the invention, and which are susceptible of modification of form, size, arrangement of parts and details of operation. The invention rather is intended to encompass all such modifications which are within its spirit and scope as defined by the claims.

What is claimed:

1. A method for positioning a trocar in a body cavity, comprising the steps of:
positioning a guide wire through a body cavity wall into a body cavity;
positioning a trocar over said guide wire; and
sliding said trocar over said guide wire and through said body cavity wall, whereby said trocar is guided by said guide wire.

2. The method of claim 1, wherein said guide wire has a resilient J-shaped tip.

3. The method of claim 1, wherein said guide wire has a braided tip.

4. The method of claim 1, wherein said guide wire has a blunt tip.

5. The method of claim 1, further comprising the steps of inserting an access needle through said body cavity wall into said body cavity, insufflating said body cavity through said access needle, and wherein said step of positioning said guide wire comprises positioning of said guide wire through said access needle into said body cavity.

6. The method of claim 5, wherein said step of insufflating said body cavity provides an insufflated body cavity, and wherein said step of positioning said guide wire comprises positioning said guide wire in said insufflated body cavity.

7. The method of claim 1, wherein said trocar is connected to a cannula and wherein said positioning step comprises positioning said trocar and said cannula over said guide wire.

8. The method of claim 7, wherein said sliding step comprises sliding said trocar and said cannula over said guide wire.

9. The method of claim 8, further comprising the step of removing said trocar from said body cavity so as to leave said cannula positioning through said body cavity wall into said body cavity.

10. A trocar assembly, comprising:
a flexible guide wire;
a trocar having a piercing tip and adapted to slide over said guide wire; and
a cannula adapted for releasable connection to said trocar.

11. The assembly of claim 10, wherein said guide wire has a resilient J-shaped tip.

12. The assembly of claim 10, wherein said guide wire has a braided tip.

13. The assembly of claim 10, wherein said guide wire has a blunt tip.

14. The assembly of claim 10, wherein said trocar comprises an elongate member having a piercing tip, and having a channel positioned along said elongate member and passing through said piercing tip, said channel being adapted to slidably receive said guide wire.
